(19) United States
(12) Patent Application Publication
(10) Pub. No.: US 2006/0258880 A1
(43) Pub. Date: Nov. 16, 2006

(54) Esterification catalyst and process for the esterification of acids in a hydrocarbons containing feed

(76) Inventor: Franciscus Hermanus Antonius Bolder, Sasolburg (ZA)

Correspondence Address: HAHN AND MOODLEY, LLP, P.O. BOX 52050, MINNEAPOLIS, MN, 55402, US

(21) Appl. No.: 10/542,962

(22) PCT Filed: Jan. 22, 2004
(86) PCT No.: PCT/ZA04/00007

§ 371 Date: Apr. 25, 2006

(30) Foreign Application Priority Data
Jan. 22, 2003 (ZA) 2003/0585

(51) Int. Cl. C07C 69/00

(52) U.S. Cl. 560/129; 208/177

(57) ABSTRACT

An esterification catalyst and process for the reduction of acids in a hydrocarbon containing composition, said process including contacting the hydrocarbon containing composition with an esterification catalyst at esterfication temperature and pressure. The esterification catalyst includes metal oxides which include one or more oxides selected from molybdenum oxide, tungsten oxide and transition metal oxides in group Ib to VIIIb.

### FIELD OF THE INVENTION

The invention provides an esterification catalyst and an esterification process for the esterification of acids in a hydrocarbons containing feed stream.

### BACKGROUND TO THE INVENTION

Fischer-Tropsch (FT) product streams are known to contain organic acids, carbonyls, alcohols and other oxygenates, but no sulphur compounds. Removing acids from FT products would allow these products to be hydrogenated at lower temperatures over nickel or other catalyst without introducing sulphur to the process. Removal of acids upstream of the refinery would also reduce the problem of corrosion which is exacerbated by the presence of water in the hydrocarbon streams.

### SUMMARY OF THE INVENTION

According to a first aspect of the invention, there is provided an esterification catalyst including one or more catalytically active metal oxides.

The metal oxides may include one or more oxides selected from transition metal oxides in group Ib to VIIIb, for example molybdenum oxide or tungsten oxide.

The molybdenum, the tungsten, or the transition metal oxide of the catalyst may be supported on a substrate.

The substrate may be alumina, silica-alumina, silica or any other suitable substrate.

According to a second aspect of the invention there is provided an esterification process for the reduction of acids in a hydrocarbon containing composition, said process including contacting the hydrocarbon containing composition with an esterification catalyst at esterfication temperature and pressure.

The esterification catalyst may be a catalyst substantially as described above.

The esterification catalyst may be a catalyst selected from the group of transition metal oxides in group Ib to VIIIb on alumina substrate, including molybdenum oxide on alumina catalyst and tungsten oxide on alumina catalyst.

The esterification temperature may be from 100° C. to 320° C.

The esterification temperature may be from 170° C. to 250° C.

Typically the esterification temperature is from 190° C. to 210° C.

The esterification pressure may be from atmospheric pressure to 100 Bar, typically from 1 to 55 Bar.

The hydrocarbon containing composition may include hydrocarbons of less than 24 carbons i.e. lower than C₂₄.

The hydrocarbon containing composition may be a C₄ to C₂₀ hydrocarbons containing composition.

The hydrocarbon containing composition may be a Fischer-Tropsch (FT) condensate fraction.

By FT condensate fraction is meant a condensate fraction of the Fischer-Tropsch reaction products. The condensate fraction is typically obtained as the light stream or overhead stream from a separator after a Fischer-Tropsch reactor in which the Fischer-Tropsch reaction has taken place. Table A below provides typical data for the FT condensate stream.

TABLE A
Typical Fischer-Tropsch product after separation
into two fractions (vol % distilled)

| | | FT Condensate | FT Wax |
|---|---|---|---|
| | | (<270° C. fraction) | (>270° C. fraction) |
| C₅-160° | C. | 44 | 3 |
| 160-270° | C. | 43 | 4 |
| 270-370° | C. | 13 | 25 |
| 370-500° | C. | | 40 |
| >500° | C. | | 28 |

The hydrocarbon condensate fraction may be a distilled fraction from the FT condensate fraction. An example of such distilled fraction is shown in table B.

TABLE B

| Carbon number | Mass % |
|---|---|
| <C₁₃ | 1.4 |
| C₁₃ | 43.8 |
| C₁₄ | 47.2 |
| >C₁₄ | 7.6 |

The hydrocarbon containing composition may have an acid level of 0.5 mg KOH/g or higher.

Typically, the acid level in the hydrocarbon containing composition may be as high as 12 mg KOH/g.

The alcohol to acid ratio in the FT hydrocarbon may be between 9 and 92 on a molar basis.

Methanol or another alcohol may be added to the FT hydrocarbon feed to increase the alcohol to acid ratio.

The product of the process may have an acid level of less than or equal to 0.5 mg KOH/g, generally from 0.1 mg KOH/g to 0.3 mg KOH/g.

The process may be carried out in a continuous flow reactor, like a trickle bed or a flooded bed reactor. The process may also be carried out in a batch reactor.

The process may be carried out at an LHSV of from 0.1 to 5 h⁻¹.

The process may be carried out at an LHSV of from 0.5 and 2 h⁻¹.

### EXAMPLES OF PERFORMING THE INVENTION

The examples that follow are not intended to limit the scope of the invention and are by way of illustration of the invention only.

Catalysts and Operating Procedures

A commercial molybdenum on alumina catalyst from BASF (M8-30) was used for experiments 1 to 5. The catalyst is produced in 5 mm diameter extrudates. The extrudates were crushed and sieved between 0.5 and 2.83 mm and diluted 1:1 with carborundum (0.5-2 mm).

TABLE 1
catalyst composition

| | MoO₃/Al₂O₃ | BASF ™ M8-30 | | |
|---|---|---|---|---|
| component | Na2O | MoO3 | Al2O3 | Total |
| Mass % | 0.07 | 15.61 | 83.87 | 100.00 |

The catalyst was dried in situ in a hydrogen flow at 125° C. and pretreated with hydrogen either at 470° C. for 10 hours or at 250° C. for 5 hours or heated to operating temperature. The temperature programmed reduction (TPR) shows a reaction with hydrogen around 430° C.

In experiment 6 an extruded WO₃/Al₂O₃ catalyst containing about 20% tungsten oxide was used which was ground to a particle size between 0.5 and 1 mm.

The experiments 1 to 4 were carried in a 27.5 mm ID bench scale reactor with a total length of 1.5 meter. Bed temperatures were measured with 6 thermocouples axially spaced inside a 6 mm OD thermocouple sheath. The reactor was operated in the down flow mode, at 55 bar, 0.56 to 1.5 V(lcat.h) liquid hourly space velocity (LHSV) and between 385 and 500 lₙ(lcat.h) hydrogen GHSV.

Experiments 5 and 6 were carried out in microreactor with an internal diameter of 12 mm. Bed temperatures were measured with 2 thermocouples axially spaced inside a 3 mm OD thermocouple sheath. The reactor was operated in the down flow mode at 5 bar and 0.56 to 0.67 l/(lcat.h) liquid hourly space velocity (LHSV) and between 300 and 450 lₙ/(lcat.h) hydrogen GHSV.

### EXAMPLES

### Example 1

The feed in the first experiment consisted of a C₄-C₂₀ Fischer-Tropsch product cut. The hydrocarbon product was passed through a caustic wash which reduced the acids to about 0.5 mg KOH/g. The molar ratio of alcohols to acids in the hydrocarbon feed was 92.

The catalyst was pretreated in hydrogen at 470° C.

The results are shown in table 3 below

TABLE 3
catalyst 1: treated at 470° C.

| T | ° C. | 250 | 210 | 190 | 210 |
|---|---|---|---|---|---|
| LHSV | h-1 | 1.5 | 1.5 | 1.5 | 1.5 |
| feed | | | | | |
| ratio | | 92 | 92 | 92 | 16.5 |
| alcohol/acid | | | | | |
| acid | mgKOH/g | 0.38 | 0.38 | 0.38 | 2.33 |
| carbonyl | mass % as MEK | 0.32 | 0.32 | 0.32 | 0.11 |
| alcohol | mass % as C7 | 6.96 | 6.96 | 6.96 | 7.74 |
| ester | mgKOH/g | 0.82 | 0.82 | 0.82 | 0.68 |
| olefins | g Br/100 g | 47.8 | na | 47.8 | 42.9 |
| product | | | | | |
| acid | mgKOH/g | 0.01 | 0.01 | 0.01 | 0.02 |
| carbonyl | mass % as MEK | 0 | 0.022 | 0.09 | 0.18 |
| alcohol | mass % as C7 | 0 | 0.64 | 6.47 | 6 |
| ester | mgKOH/g | 0 | 1.2 | 1.53 | 2.97 |
| olefins | g Br/100 g | 80.8 | na | 45.8 | 40.8 |
| acids | % | 97.37 | 97.37 | 97.37 | 99.14 |
| conversion | | | | | |

Fresh MoOₓ/Al₂O₃ catalyst showed initially considerable catalytic activity towards both esterification and dehydrogenation/dehydration reactions. At the beginning of the run all oxygenates were removed at 250° C. and the olefins concentration doubled from 40 to 80 gBr/100 g. The latter reactions were however short lived. The residual acids in the effluent stream was 0.01 mg KOH/g.

In the same experiment, in the temperature range of 190 to 210° C., the acids still reacted nearly completely to esters but less of the other oxygenates reacted and over time side reactions decreased.

At the lower temperatue the olefinity of the effluent was similar to the value of the feed.

### Example 2

In this example the feed consisted of C₄ to C₂₀ paraffin with a higher acid level (2.3 mg KOH/g). The alcohol concentration was the same as in the previous feed, about 7 mass % as C₇ alcohol. The molar ratio of alcohol to acid of this feed was 16.5.

The catalyst treatment was the same as in example 1.

The results are shown in table 3 above.

The residual acids in the effluent were between 0.02 and 0.03 mgKOH/g at 210° C. When the temperature was increased to 250° C. (after about 5 days), the residual acid level increased to 0.15 mg KOH/g. This may be ascribed to a decrease of the alcohol concentration due to secondary reactions. As a result the alcohol to acid ratio decreased causing a decrease in the conversion to esters.

After 3 weeks on line the oxygenates could no longer be removed at a temperature of 250° C. The temperature had to be increased to 310° C. before the bulk of the oxygenates was removed.

### Example 3

In this experiment the molybdenum oxide on alumina catalyst was pretreated in hydrogen at 250° C.

The feed for this experiment was a light condensate fraction derived from low-temperature Fischer-Tropsch synthesis (mainly in the naphtha and diesel range with a small fraction waxy material suspended in it). The acids varied between 1.9 and 2.5 mg KOH/g.

The results are shown in table 4 below

At a temperature of 210° C., 1 h⁻¹ LHSV and an alcohol to acid ratio of 14 the conversion of the acids was 98.8% resulting in an acid number of 0.03 mg KOH/g in the effluent. The same results were obtained at 1.5 h⁻¹ LHSV, which indicated that the reaction was close to equilibrium.

The stability of the catalyst was tested at 220° C. for 13 days at 1 h⁻¹ LHSV with a different feed (alcohol to acid ratio of 19). The residual acids were 0.05 mgKOH/g and remained stable for as long as these conditions were maintained.

At temperatures between 250 and 290° C. the acid content of the product increased and only at 310° C. did they decrease. Significantly, the olefinity of the product did not increase at these temperatures.

TABLE 4
catalyst 2: treated at 250° C.

| T | ° C. | 210 | 210 | 220 | 230 | 250 | 290 | 310 |
|---|---|---|---|---|---|---|---|---|
| LHSV | h-1 | 1 | 1.5 | 1 | 1 | 1.5 | 1.5 | 1.5 |
| feed | | | | | | | | |
| ratio alcohol/acid | | 14 | 14 | 19 | 19 | 14 | 14 | 14 |
| acid | mgKOH/g | 2.5 | 2.5 | 1.9 | 1.9 | 2.5 | 2.5 | 2.5 |
| carbonyl | mass % as MEK | 0.46 | 0.46 | 0.4 | 0.4 | 0.46 | 0.46 | 0.46 |
| alcohol | mass % as C7 | 7 | 7 | 7.4 | 7.4 | 7 | 7 | 7 |
| ester | mgKOH/g | 0.75 | 0.75 | 0.97 | 0.97 | 0.75 | 0.75 | 0.75 |
| olefins | g Br/100 g | 66.6 | 66.6 | 68.3 | 68.3 | 66.6 | 66.6 | 66.6 |
| product | | | | | | | | |
| acid | mgKOH/g | 0.03 | 0.03 | 0.05 | 0.09 | 0.19 | 0.6 | 0.03 |
| carbonyl | mass % as MEK | 0.26 | 0.31 | 0.21 | 0.21 | 0.24 | 0.29 | 0.08 |
| alcohol | mass % as C7 | 6.3 | 7.2 | 3.6 | 3.4 | 3 | 0.68 | 0.18 |
| ester | mgKOH/g | 3.2 | 3.4 | 2.6 | 2.6 | 3.3 | 0.59 | 0.05 |
| olefins | g Br/100 g | 65.0 | 65.0 | 71.2 | 63.2 | 64.2 | 67.7 | 66.0 |
| conversion | | | | | | | | |
| acids conversion | % | 98.80 | 98.80 | 97.37 | 95.26 | 92.40 | 76.00 | 98.80 |

Carbonyls were only partly removed and the temperature made little difference to the conversion.

Depending on the temperature, alcohols and carbonyls may react to form a range of compounds and a change in the alcohol to acid ratio will shift the equilibrium from ester to free acids.

Apart from esterification, alcohols can undergo a variety of other reactions:
aldol condensation with aldehydes
acetal and ether formation
dehydration to olefins

There was insufficient evidence to conclude that alcohols were dehydrated to olefins because the olefin level did not increase consistently. Temperatures of well above 300° C. are required to dehydrate significant amounts of ethanol and propanol to the corresponding olefins.

### Example 4

The feed in this experiment consisted of a C₁₀-C₁₃ Fischer Tropsch product cut. The hydrocarbon product was high in acids (about 12.5 mg KOH/g) and contained other oxygenates. Methanol was co-fed with the Fischer-Tropsch product at such ratio that the molar ratio of alcohols to acids in the hydrocarbon feed was equal to 10.

The catalyst was molybdenum oxide on alumina which was pretreated in hydrogen at 250° C.

The results are shown in table 5 below.

TABLE 5

| LHSV | h-1 | 1 | 1 | 0.56 | 0.56 |
|---|---|---|---|---|---|
| Pressure | bar | 40 | 40 | 40 | 5 |
| Temp) | ° C. | 200 | 220 | 220 | 220 |
| Ethanol | g/kg feed | 141 | 141 | 141 | 141 |
| alcohol/acid | | 23.3 | 23.3 | 26.1 | 26.1 |
| SLO feed | | | | | |
| acid | mgKOH/g | 10.5 | 10.5 | 10 | 10 |
| carbonyl | mass % | 2.66 | 2.66 | 2.96 | 2.96 |
| | as MEK | | | | |
| alcohol | mass % | 50.5 | 50.5 | 53.9 | 53.9 |
| | as C7 | | | | |
| ester | mgKOH/g | 5.3 | 5.3 | 3.7 | 3.7 |
| Product | | | | | |
| acids | mgKOH/g | 2 | 0.75 | 0.34 | 0.27 |
| carbonyl | mass % | 2.4 | 2.14 | 1.6 | 1.6 |
| | as MEK | | | | |
| alcohol | mass % | 25 | 22.4 | 27 | 27 |
| | as C7 | | | | |
| ester | mgKOH/g | 12.9 | 16.2 | 15.8 | 15.8 |
| acid | | | | | |
| conversion | % | 81.0 | 92.9 | 96.6 | 97.3 |

SLO: Stabilised Light Oil

### Example 5

The catalyst was the same as used in experiment 1, but the reaction was carried out in a microreactor. 20 ml Catalyst with a particle size between 0.5 and 1.0 mm was loaded. The catalyst was heated up in a hydrogen stream to the reaction temperature at which point the feed was introduced. The feed consisted of a C₁₃-C₁₄ FT hydrocarbon product fraction with an acid number of 12.6 mg KOH/g. Methanol was co-fed with the FT hydrocarbon. The ratio of alcohol to acid in the mixture was between 9 and 12 on a molar basis. The reaction was carried out at 0.67 h⁻¹ LHSV (hydrocarbon feed), 190 h⁻¹ hydrogen GHSV and 5 bar g pressure.

A reduction of the acids in the hydrocarbon from 12.6 to 0.3 mg KOH/g was achieved in the temperature range of 210 to 230° C. This amounts to a conversion of 97.7% of the acids to esters in a single pass.

### Example 6

The catalyst in this example was tungsten oxide on alumina, containing about 20 mass % WO₃.

The equipment and the experimental conditions were the same as described in example 5.

Similar to the previous example, the catalyst was heated up in a hydrogen stream to reaction temperature and the feed introduced.

The results are shown in table 6. The acid concentration in the hydrocarbon was reduced to 0.3 mg KOH/g at 5 bar g, 210° C., 0.71 h⁻¹ LHSV and an alcohol/acic ration of 17.

TABLE 6

| Temp | deg C. | 210 | 220 | 230 | 240 | 190 | 210 |
|---|---|---|---|---|---|---|---|
| Pres | barg | 5 | 5 | 5 | 5 | 5 | 5 |
| H2 flow | % | 30 | 30 | 30 | 30 | 30 | 30 |
| LHSV | | 0.67 | 0.67 | 0.67 | 0.67 | 0.71 | 0.71 |
| Alc/Acid | mol/mol | 8.95 | 8.95 | 8.95 | 8.95 | 17.33 | 17.33 |
| FEED | | | | | | | |
| Acid | mgKOH/g | 12.6 | | | | | |
| Carbonyl | mass % as CO | 1.4 | | | | | |
| Alcohol | mass % as C7 | 3.1 | | | | | |
| Ester | mgKOH/g | 7.2 | | | | | |
| PRODUCT | | | | | | | |
| Acid | mgKOH/g | 0.34 | 0.43 | 0.49 | 0.68 | 0.55 | 0.29 |
| Carbonyl | mass % as CO | 0.85 | 1.1 | 1.1 | 1.1 | 1.2 | 0.95 |
| Alcohol | mass % as C7 | 2 | 1 | 1.6 | 1.1 | 3 | 3.7 |
| Ester | mgKOH/g | 14.6 | 11.9 | 12.6 | 12.1 | 15 | 14.4 |

1. An esterification catalyst including one or more catalytically active metal oxides.

2. An esterification catalyst as claimed in claim 1, wherein the metal oxides include one or more oxides selected from transition metals in group Ib to VIIIb.

3. An esterification catalyst as claimed in claim 1, wherein the metal oxide consists of molybdenum oxide or tungsten oxide.

4. An esterification catalyst as claimed in claims 1 to 3, wherein the molybdenum, the tungsten, or any other transition metal oxide is supported on a substrate.

5. An esterfication catalyst as claimed in claim 4, wherein the substrate is alumina, silica-alumina, or silica.

6. An esterification process for the reduction of acids in a hydrocarbon containing composition, said process including contacting the hydrocarbon containing composition with an esterification catalyst at esterfication temperature and pressure.

7. An esterification process as claimed in claim 6, wherein the esterification catalyst is a catalyst as claimed in claims 1 to 5.

8. An esterification process as claimed in claim 6 or claim 7, wherein the esterification catalyst is a catalyst selected from the group of transition metal oxides in group Ib to VIIIb on alumina catalyst, including molybdenum oxide on alumina catalyst and tungsten oxide on alumina catalyst.

9. An esterification process as claimed in any one of claims 6 to 8, wherein the esterification temperature is from 100° C. to 320° C.

10. An esterification process as claimed in claim 9, wherein the esterification temperature is from 170° C. to 250° C.

11. An esterification process as claimed in claim 10, wherein the esterification temperature is from 190° C. to 210° C.

12. An esterification process as claimed in any one of claims 6 to 11, wherein the esterification pressure is from atmospheric pressure to 100 Bar.

13. An esterification process as claimed in claim 12, wherein the esterification pressure is from 1 to 55 Bar.

14. An esterification process as claimed in any one of claims 6 to 13, wherein the hydrocarbon containing composition includes hydrocarbons of less than 24 carbons i.e. lower than C₂₄.

15. An esterification process as claimed in any one of claims 6 to 13, wherein the hydrocarbon containing composition is a C₄ to C₂₀ hydrocarbons containing composition.

16. An esterification process as claimed in any one of claims 6 to 15, wherein the hydrocarbon containing composition is a Fischer-Tropsch (FT) condensate fraction.

17. An esterification process as claimed in claim 16, wherein the hydrocarbon condensate fraction is a distilled fraction from the FT condensate fraction.

18. An esterification process as claimed in any one of claims 6 to 17, wherein the hydrocarbon containing composition has an acid level of 0.5 mg KOH/g or higher.

19. An esterification process as claimed in claim 18, wherein the acid level in the hydrocarbon containing composition is up to 12 mg KOH/g.

20. An esterification process as claimed in any one of claims 16 to 19, wherein the alcohol to acid ratio in the FT hydrocarbon is between 9 and 92 on a molar basis.

21. An esterification process as claimed in any one of claims 16 to 20, wherein methanol or another alcohol is added to the FT hydrocarbon feed to increase the alcohol to acid ratio.

22. An esterification process as claimed in any one of claims 6 to 21, wherein the product of the process has an acid level of less than or equal to 0.5 mg KOH/g.

23. An esterification process as claimed in any one of claims 6 to 22, wherein the product of the process has an acid level of from 0.1 mg KOH/g to 0.3 mg KOH/g.

24. An esterification process as claimed in any one of claims 6 to 23, wherein the process is carried out in a continuous flow reactor.

25. An esterification process as claimed in any one of claims 6 to 23, wherein the process is carried out at an LHSV of from 0.1 to 5 h⁻¹.

26. An esterification process as claimed in any one of claims 6 to 25, wherein the process is carried out at an LHSV of from 0.5 to 2 h⁻¹.
